(19) United States
(12) Patent Application Publication
(10) Pub. No.: US 2010/0234751 A1
(43) Pub. Date: Sep. 16, 2010

(54) MODULAR KIT OF COMPLEMENTARY PLETHYSMOGRAPHIC APPARATUS COMPONENTS

(75) Inventors: Thomas Florian SCHUESSLER, Ville Mont-Royal (CA); Ilan Benjamin UROVITCH, Cote-Saint-Luc (CA)

Correspondence Address: CHOATE, HALL & STEWART LLP, TWO INTERNATIONAL PLACE, BOSTON, MA, 02110, US

(73) Assignee: SCIREQ SCIENTIFIC RESPIRATORY EQUIPMENT INC., Montreal (CA)

(21) Appl. No.: 12/723,958

(22) Filed: Mar. 15, 2010

Related U.S. Application Data
(60) Provisional application No. 61/159,888, filed on Mar. 13, 2009.

(51) Int. Cl. A61B 5/097

(52) U.S. Cl. 600/534

(57) ABSTRACT

A modular kit of complementary plethysmographic apparatus components including at least one tube, at least one termination device for closing an open extremity of the tube and first and second complementary coupling mechanisms formed on the tube and the termination device. The coupling mechanisms are adapted to provide sealing engagement between the tube and the termination device. The first and second complementary coupling mechanisms comprise a notched interface component adapted to fit into a corresponding mating gap component, such that a rotation of the two components with respect to each other, solidly locks the two components together while applying pressure onto a face seal between the two components.

### FIELD OF THE INVENTION

The present invention generally relates to a scientific apparatus for the measurement of lung function in laboratory animals as used in pre-clinical medical and pharmacological research. More particularly, the present invention relates to a modular kit of complementary plethysmographic apparatus components.

### BACKGROUND OF THE INVENTION

There is currently a wide variety of documented apparatuses and methods to measure lung function in laboratory animals such as mice, rats and guinea pigs. These methods possess different levels of invasiveness and differ in the sensitivity and specificity of the measurements they produce.

One approach to measuring breathing waveforms is to place a subject in a closed chamber referred to as a plethysmograph. The most common plethysmographs are elongated, tubular structures with mostly round cross sections in which an animal can be placed such that the nose-to-tail axis of the subject's body is aligned with the center axis of the tube. Both ends of the tube are closed either permanently or via sealed attachment against a face plate, although at least one side must allow opening to permit insertion of the subject. The setups further contain ports to measure pressures or flows and/or to pass catheters to the subject.

Depending on the application, tubular plethysmograph designs may be used to:
(i) contain a conscious, spontaneously breathing subject with enough room that the respiratory motion is not restricted, permitting indirect measures of lung function to be obtained from the pressure inside and/or flow into and out of the plethysmograph (Unrestrained Whole Body Plethysmography, UWBP);
(ii) contain an anaesthetised, tracheally intubated or tracheotomised subject with the tracheal cannula connected to the outside of the box, so that airflow corresponding to chest wall expansion can be measured (Anaesthetised Whole Body Plethysmography, AWBP);
(iii) contain and restrain a subject with its airway openings protruding to the outside of the box through a nose or neck seal, so that airflow corresponding to chest wall expansion can be measured (Head-out Plethysmography, HOP);
(iv) contain and restrain a subject with its airway openings protruding into a second chamber through a nose or neck seal, so that both the airflow at the airway opening and the airflow corresponding to chest wall expansion can be measured independently, permitting the calculation of specific airway resistance (Double-chamber Plethysmography, DCP);
(v) contain and restrain a subject with its airway openings protruding through a nose or neck seal into an inhalation exposure tower or chamber, so that exposure to inhaled toxins or compounds can be limited to the nose or head, and the airflow corresponding to chest wall expansion can be optionally measured (Plethysmographic Restraint).

The raw data waveforms from these plethysmographs are commonly analyzed further, possibly in conjunction with additional data collected independently of the plethysmograph, to calculate physiologically meaningful parameters of lung function such as tidal volume, respiratory rate, airway resistance, lung compliance or various lung volumes.

Prior art documents known to the Applicant include: U.S. Pat. No. 5,379,777; U.S. Pat. No. 5,379,778; U.S. Pat. No. 6,902,532; and U.S. Pat. No. 7,402,137 B2.

With the exception of head-out plethysmographs commonly being extended to double-chamber plethysmographs, there is, to the Applicant's knowledge, no unifying design that permits the reuse of tubular components for all different types of plethysmographs listed above.

### SUMMARY OF THE INVENTION

An object of the present invention is to provide a modular kit of complementary plethysmographic apparatus components that satisfies the above-mentioned need.

According to the present invention, there is provided a modular kit of complementary plethysmographic apparatus components, comprising:
at least one tube;
at least one termination device for closing an open extremity of the at least one tube; and
first and second complementary coupling mechanisms formed on the at least one tube and the at least one termination device, the coupling mechanisms being adapted to provide sealing engagement between the at least one tube and the at least one termination device,
   wherein the first and second complementary coupling mechanisms comprise a notched interface component adapted to fit into a corresponding mating gap component, such that a slight rotation of the two components with respect to each other, solidly locks the two components together while applying pressure onto a face seal between the two components.

### BRIEF DESCRIPTION OF DRAWINGS

These and other objects and advantages of the invention will become apparent upon reading the detailed description and upon referring to the drawings in which:

FIG. 1 is a perspective view of a modular kit of complementary plethysmographic apparatus components according to a preferred embodiment of the present invention.

FIG. 2 is a perspective view of a lock-and-seal mechanism for use with the kit shown in FIG. 1.

FIG. 3 is a cross-sectional side view of the lock-and-seal mechanism shown in FIG. 2.

FIG. 4 is a perspective view of the tube components of the kit shown in FIG. 1.

FIG. 5 is a perspective view of the tube adapters of the kit shown in FIG. 1.

FIG. 6 is a perspective view of the end caps of the kit shown in FIG. 1.

FIG. 7 is a perspective view of a height adjustable tubing port for an end cap according to a preferred embodiment of the present invention.

FIG. 8 is a cross-sectional side view of a restraining mechanism inside a plethysmograph chamber, in an unlocked position, in accordance with a preferred embodiment of the present invention (with a removable handle not shown).

FIG. 9 is a cross-sectional side view of the restraining mechanism shown in FIG. 8, in a locked position (with the removable handle not shown).

### DETAILED DESCRIPTION OF PREFERRED EMBODIMENTS

Although the invention is described in terms of specific embodiments, it is to be understood that the embodiments described herein are by way of example only and that the scope of the invention is not intended to be limited thereby.

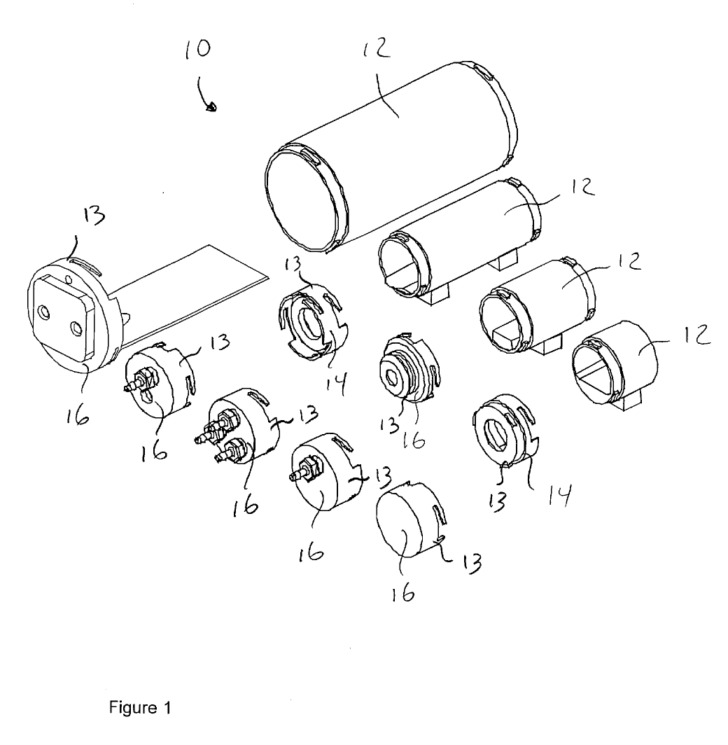

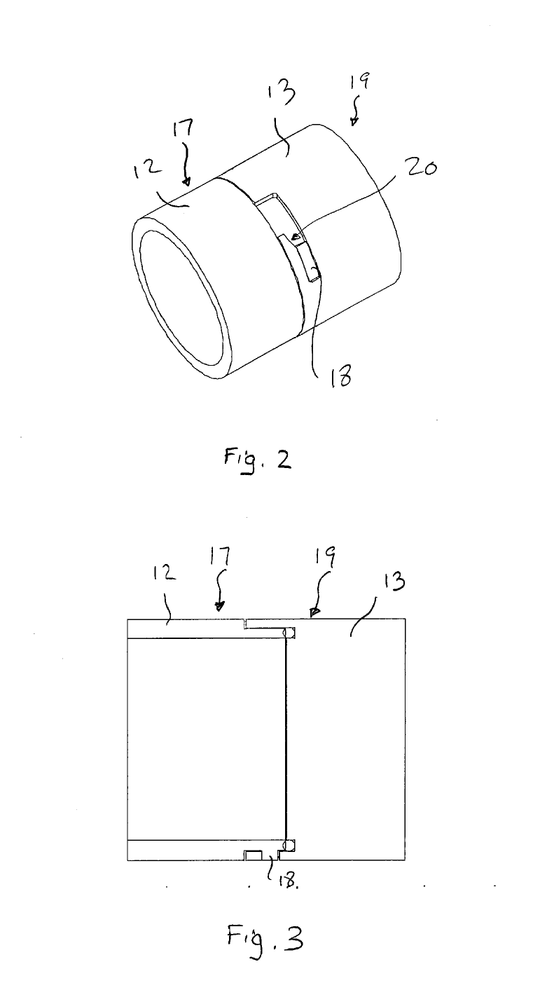

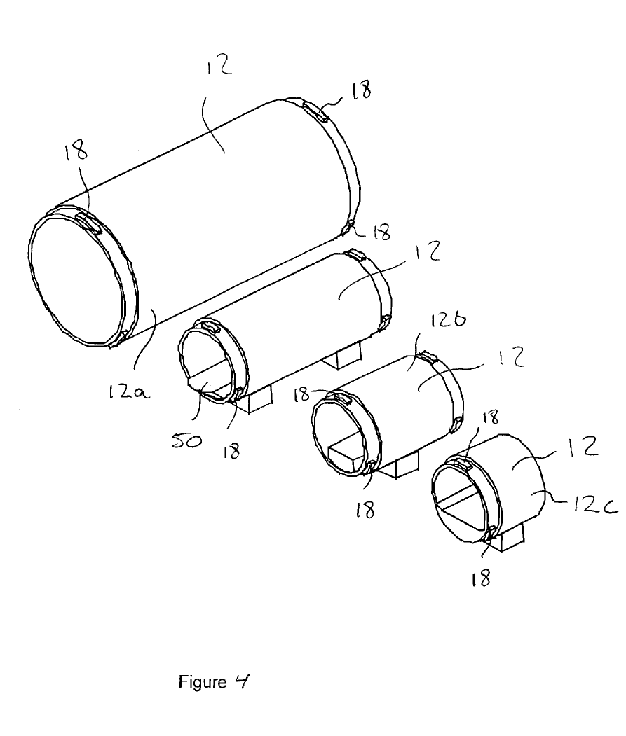

As shown in FIG. 1, according to the present invention, there is provided a modular kit 10 of complementary plethysmographic apparatus components. The kit 10 includes a plurality of tubes 12 of varying sizes and a plurality of termination devices 13 for closing an open extremity of the tubes 12. As better shown in FIGS. 2 and 3, first and second complementary coupling mechanisms 17, 19 are formed on the tubes 12 and the termination devices 13. The coupling mechanisms are adapted to provide sealing engagement between the tube 12 and the termination device 13. The first and second complementary coupling mechanisms comprise a notched interface component 18 adapted to fit into a corresponding mating gap component 20, such that a slight rotation of the two components with respect to each other, solidly locks the two components together while applying pressure onto a face seal between the two components.

Although in general, two termination devices are typically used to close opposite open extremities of tubes, the kit in accordance with the present invention may also be used with tubes that each have only one open extremity, with the opposite extremity being permanently closed or sealed.

In prior art systems, three types of seals have been employed to provide seals for plethysmograph chambers: i) face seals; using luggage clamps or thumb screws to apply pressure on the seal in an axial direction; ii) rubber cap-style seals; or iii) radial seals (see for example, U.S. Pat. No. 5,379,778). The first two designs are unpractical, cumbersome and industrial-looking, while the third design, although deemed self-supporting, can support very limited weight and may easily be dislodged during use.

As better shown in FIGS. 2 and 3, the Applicant has conceived a design that pairs a face seal with a bayonet clamping mechanism to provide an integrated lock-and-seal mechanism that combines i) reliable seal; ii) robust mechanical attachment; and iii) great ease of use. As mentioned above, the ends of the tubes 12 are shaped to have notches 18 that can fit into a mating gap 20 in the termination devices 13, including the end caps 16 and tube adapters 14. Once the two parts are connected, a slight rotation of the tube with respect to its mating piece solidly locks the two parts together and provides additional pressure on the face seal, ensuring a tightly sealed connection.

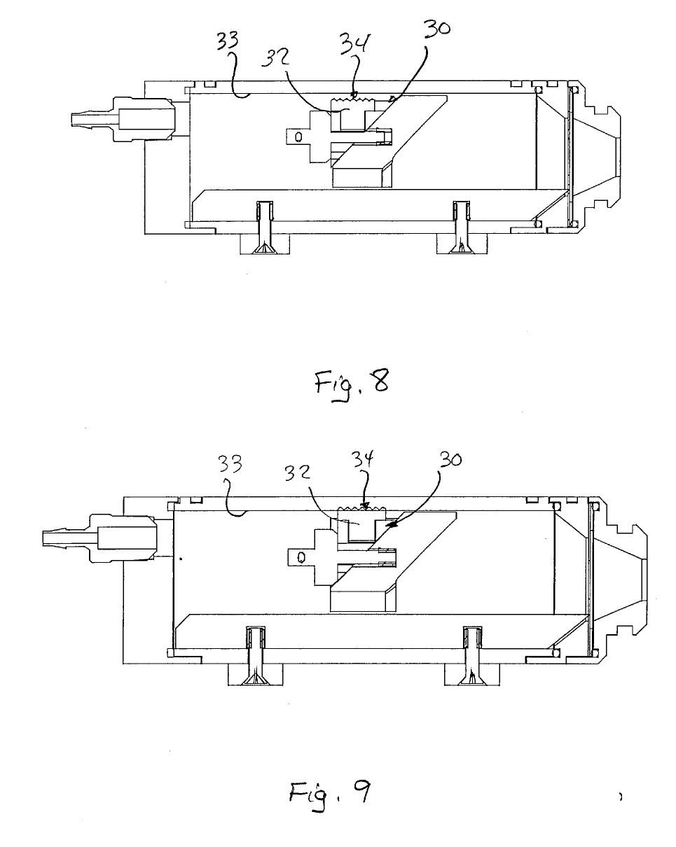

The present invention consists of a modular design that consists of a variety of tubes 12 and termination devices 13, including tube adapters 14 and end caps 16, with different sizes, shapes and features. These components can be combined in a variety of ways to create the above-mentioned types of plethysmographs for different sizes of subjects. Tubes 12, adapters 14 and end caps 16 connect through a novel coupling or sealing mechanism that is quick and easy to attach yet sure to remain securely attached and aligned even in the presence of vibrations of accidental mechanical shock. Where needed, a new, compact restraint mechanism, as shown in FIGS. 8 and 9 is used to permit easy, reliable restraint of the subject without compromising the chamber seal or requiring large chamber volumes.

The modular design concept includes the following components, each of which may exist in various sizes to accommodate different size subjects.

### Tubes

As better shown in FIG. 4, there are various types of tube components 12, including:
i) Long tubes 12a for subject body;
ii) Short tubes 12b for use as head chambers;
iii) Partial or open tubes 12c that may act as head rests and/or to restrict the subject's vision range.

The tubes have the following features:
Tube sizes are selected such that some tubes may serve different functions for different subjects.
Tubes may include a subject bed 50 to prevent rotation of the subject.
Long tubes are dimensioned as small as possible in order to ensure a good frequency response for flow measurements, but as large as necessary to permit easy insertion of subject.
Short tubes are dimensioned such that, in a double-chamber plethysmograph configuration, the net air volume with a typical subject placed in the plethysmograph is identical in the head and body chambers in order to ensure that the dynamic properties are identical on both sides.

### Tube Adapters

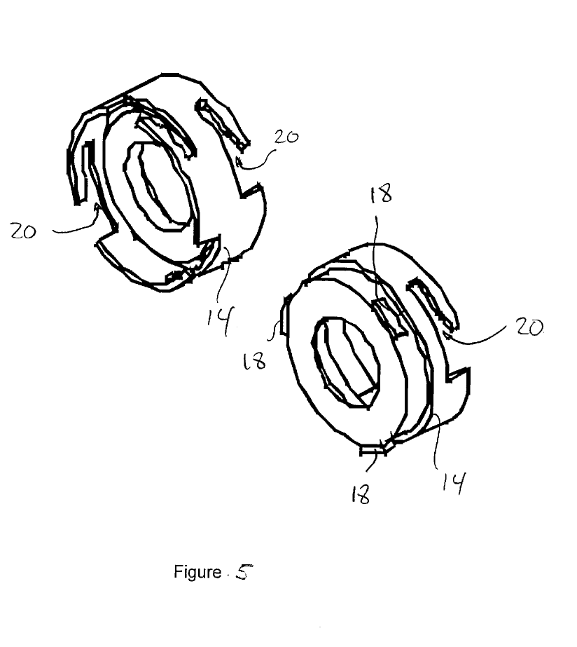

As shown in FIG. 5, the tube adapters 14 are designed to permit longitudinal connection of two or more tubes on the same axis or shifted parallel axes. Typically, tube adapters contain some form of interface that divides the device into multiple segments that perform specific functions. For example, a tube adapter 14 containing a neck or head seal may be used with two tubes to make a double-chamber plethysmograph.

### End Caps

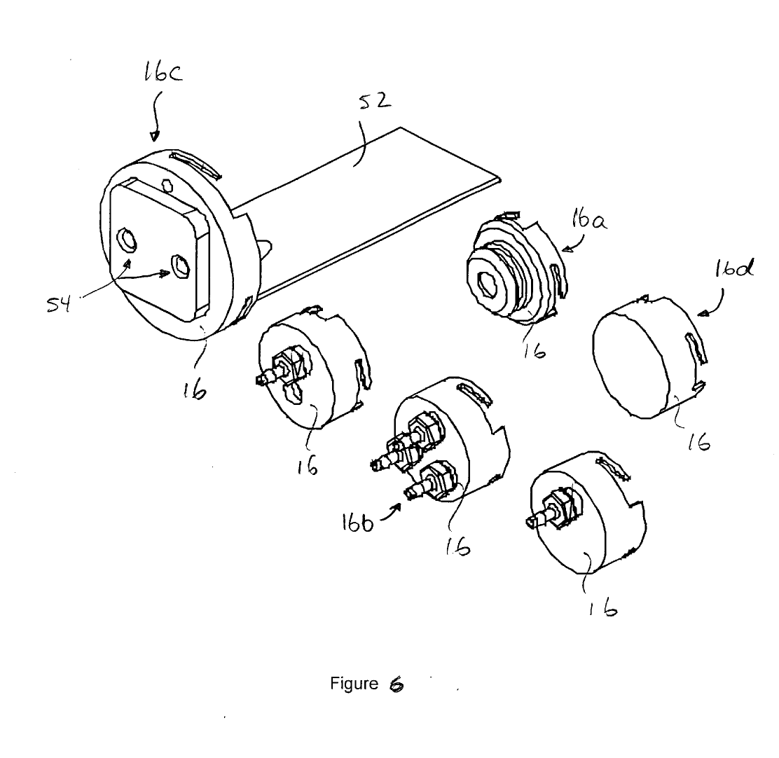

As shown in FIG. 6, there are various types of end caps 16, including:
i) Nose seal 16a with exposure tower adapter for Plethysmograph Restrainers.
ii) Closed end cap 16b with ports for cannulas, pressure or flow measurements.
iii) Closed end cap 16c with subject bed 52 and ports 54 for ventilation Y-tubing and cannulas.
iv) Fully closed version 16d.

### Adjustable Ports

When working with intubated or trachostomized animals, it is important to position the Y-tubing and cannula based on animal size in order to prevent accidental extubation or damage to the trachea. One prior art document (U.S. Pat. No. 7,402,137 B2) employs a height-adjustable subject table, while most other existing plethysmograph designs have a fixed geometry and hence do not permit adaptation of the tubing position for individual animals.

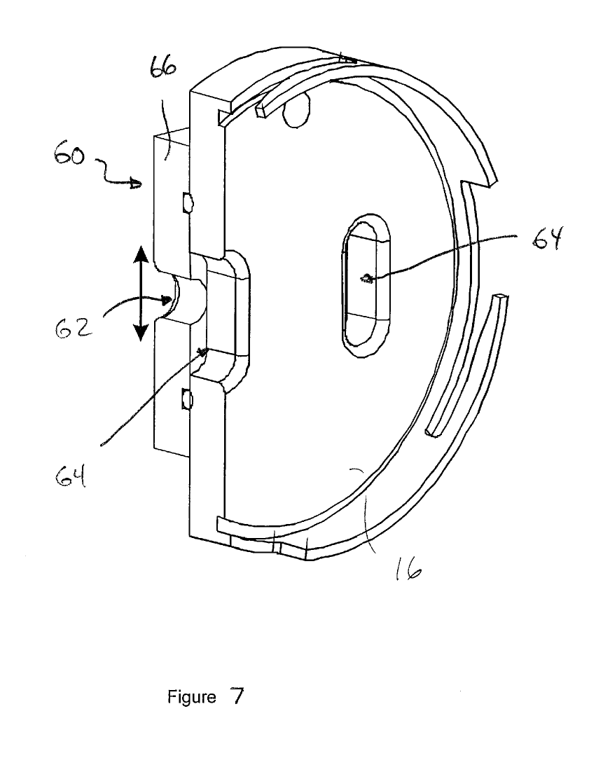

As shown in FIG. 7, one of the end caps 16 of the kit may include an end cap design for ventilated subjects which includes a subject bed that is permanently and rigidly attached to the end cap at a fixed height. A height adjustment system 60 for adjusting a height of selected ones of the plurality of external interface ports 62 of the end cap having tubing passing therethrough. Ventilation Y-tubing is passed through vertical slots 64 in the end cap 16 and held in place by a guide plate 66. The vertical position of the guide plate 66 on the end cap 16 is user adjustable, permitting adjustment of the height of the Y-tubing ports relative to the subject.

### Valved Ports

Preferably, selected ones of the plurality of external interface ports on the tubes or end caps having inspiratory and expiratory tubing passing therethrough are each fitted with a valve mechanism. The valve mechanisms are positioned proximate the subject such that, when the valve mechanisms are closed, the airways of the subject are identified or considered as being occluded.

### Restraining Mechanisms

In prior art, two mechanisms have been commonly used to restrain subjects in tubular chambers: i) restraining mechanisms guided by one or more longitudinal slots in the tube walls; and ii) mechanisms based on lead screws. The first approach provides good force feedback as needed to ensure adequate but not excessive restraining, but it is virtually impossible to seal and therefore not suitable for plethysmographs. The second approach is slow to advance, making subject placement more difficult and time consuming, provides very little force feedback and usually results in cumbersome, large setups with comparatively large internal chamber volumes and poor frequency responses.

As shown in FIGS. 8 and 9, preferably, at least one tube of the kit comprises a restraining mechanism 30. The restraining mechanism 30 utilizes a movable plunger with a long, removable handle (not shown in the figure). The plunger 32 can be rapidly positioned so that the subject can easily be captured in the ideal position. By turning the handle, the plunger is then interlocked against the inner tube surfaces 33 by means of friction, interlocking saw teeth or a similar mechanism 34, such as friction fit components. Finally, the handle is removed from the plunger and taken out of the plethysmograph, leaving the plunger in a locked position and fully contained inside the plethysmograph. This design provides excellent force feedback while positioning the plunger and permits comparatively compact plethysmograph chambers with small internal volumes and good frequency responses. In another embodiment of the present invention, a sawtooth mechanism can be provided where the handle does not need to be turned for advancing the plunger, but only to release the plunger from the sawtooth mechanism and pulling it back.

Preferably, one single tube of the kit is used to completely accommodate a sufficiently passive subject. Two closed end caps are attached to opposite ends of the single tube to form a closed plethysmographic chamber. In this configuration, a plurality of external interface ports are connected to the tube.

Preferably, the external interface ports are adapted to receive a flow or pressure measuring device. Such a configuration may be used for unrestrained whole body plethysmography.

Preferably, selected ones of the external interface ports are adapted to allow passage of cannulae into the chamber for positive pressure mechanical ventilation and/or vascular cannularisation of the subject.

The configurations of tubes with interface ports for measuring devices and cannulae can be used as plethysmographs for anaesthetised subjects.

Preferably, one of the external interface ports on one of the tubes or end caps is fitted with a pneumotachometer to measure air flow in and out of the chamber that results from respiratory chest wall motion, which may be useful for unrestrained whole body, anaesthetised whole body and head-out plethysmographic applications.

Preferably, one of the external interface ports on one of the tubes or end caps is fitted with a transducer to measure pressure changes in the chamber that reflect respiratory chest wall motion, which may be useful for unrestrained whole body plethysmographic applications.

Preferably, one of the end caps comprises a rigidly attached subject bed, a foot for providing mechanical support for the plethysmograph and a plurality of external interface ports. These elements can be used to form a plethysmograph wherein the end cap and tube can be easily and rapidly separated and refitted to each other to open and reseal the plethysmograph, without altering the instrumentation of the subject and without any tethers.

Preferably, the modular kit may comprise elements such as two longitudinally connected tubes forming first and second chambers of a double-chamber plethysmograph. A tube adapter, such as a neck seal or a nose seal, is used to link the first and second chambers. Both the airflow at the airway opening and the airflow corresponding to chest wall expansion can be measured independently in such a configuration, permitting the calculation of specific airway resistance.

Preferably, the modular kit may further comprise an inhalation exposure system connected to a tube through a tube adapter. The inhalation exposure system can be an inhalation exposure tower or an inhalation exposure chamber. Such a configuration can be used with a restraining mechanism as described above. The subject will have its nose protruding through a nose or neck seal into the inhalation exposure tower or chamber. The exposure to inhaled toxins or compounds can thus be limited to the nose or head, and the airflow corresponding to chest wall expansion can be optionally measured for plethysmographic restraint applications.

Although preferred embodiments of the present invention have been described in detail herein and illustrated in the accompanying drawings, it is to be understood that the invention is not limited to these precise embodiments and that various changes and modifications may be effected therein without departing from the scope or spirit of the present invention.

1. A modular kit of complementary plethysmographic apparatus components, comprising
at least one tube;
at least one termination device for closing an open extremity of the at least one tube; and
first and second complementary coupling mechanisms formed on the at least one tube and the at least one termination device, said coupling mechanisms being adapted to provide sealing engagement between the at least one tube and the at least one termination device,
wherein the first and second complementary coupling mechanisms comprise a notched interface component adapted to fit into a corresponding mating gap component, such that a slight rotation of the two components with respect to each other, solidly locks the two components together while applying pressure onto a face seal between the two components.

2. The modular kit according to claim 1, wherein the at least one tube comprises a restraining mechanism, the restraining mechanism comprising:
a movable plunger displaceable by a removable handle, and rapidly positionable for capturing a subject,
wherein, through a turn of the handle, the plunger is interlocked against inner tube surfaces by means of friction, interlocking an interlocking mechanism, and the handle is removable from the plunger to be taken out of the tube, for leaving the plunger in a locked position and fully contained inside the tube.

3. The modular kit according to claim 2, wherein the interlocking mechanism is selected from the group comprising saw tooth interfaces and friction fit components.

4. The modular kit according to claim 1, further comprising at least one tube adapter shaped to permit longitudinal connection of two or more tubes, said at least one tube adapter comprising a third coupling being adapted to provide sealing engagement between the at least one tube and the at least one tube adapter.

5. The modular kit according to claim 1, comprising:
one single tube used to completely accommodate a sufficiently passive subject,
wherein the at least one termination device comprises two closed end caps attached to opposite ends of the single tube to form a closed plethysmographic chamber, and the modular kit further comprises a plurality of external interface ports connected to the chamber.

6. The modular kit according to claim 5, wherein at least one of the external interface ports is adapted to receive a flow or pressure measuring device.

7. The modular kit according to claim 5, wherein selected ones of the external interface ports are adapted to allow passage of cannulae into the chamber for positive pressure mechanical ventilation and/or vascular cannularisation of the subject.

8. The modular kit according to claim 5, wherein one of said plurality of external interface ports is fitted with a pneumotachometer to measure air flow in and out of the chamber that results from respiratory chest wall motion.

9. The modular kit according to claim 5, wherein one of said plurality of external interface ports is fitted with a transducer to measure pressure changes in the chamber that reflect respiratory chest wall motion.

10. The modular kit according to claim 1, wherein the at least one termination device comprises:
a rigidly attached subject bed;
a foot for providing mechanical support for the plethysmograph; and
a plurality of external interface ports.

11. The modular kit according to claim 5, wherein the modular kit further comprises a height adjustment system for adjusting a height of selected ones of the plurality of external interface ports having tubing passing therethrough.

12. The modular kit according to claim 5, wherein selected ones of the plurality of external interface ports having inspiratory and expiratory tubing passing therethrough are each fitted with a valve mechanism.

13. The modular kit according to claim 12, wherein said valve mechanisms are positioned proximate the subject such that, when the valve mechanisms are closed, the airways of the subject are identified as being occluded.

14. The modular kit according to claim 1, wherein the termination device is a tube adapter, said tube adapter being selected from the group comprising a neck seal and a nose seal.

15. The modular kit according to claim 14, further comprising a partially open tube for use as a head rest and/or to restrict a vision range of a subject.

16. The modular kit according to claim 14, comprising:
two longitudinally connected tubes forming first and second chambers of a double-chamber plethysmograph,
wherein the tube adapter links the first and second chambers.

17. The modular kit according to claim 14, comprising one tube, further comprising:
an inhalation exposure system connected to the tube through the tube adapter, said inhalation exposure system being selected from the group comprising an inhalation exposure tower and an inhalation exposure chamber.

18. The modular kit according to claim 2, wherein the termination device is a tube adapter, said tube adapter being selected from the group comprising a neck seal and a nose seal.

19. The modular kit according to claim 18, wherein the at least one tube comprises a subject bed.

20. The modular kit according to claim 11, wherein selected ones of the plurality of external interface ports having inspiratory and expiratory tubing passing therethrough are each fitted with a valve mechanism.
